(19) United States
(12) Patent Application Publication
(10) Pub. No.: US 2021/0137676 A1
(43) Pub. Date: May 13, 2021

(54) GROWTH-ACCOMMODATING VALVE SYSTEM

(71) Applicant: THE REGENTS OF THE UNIVERSITY OF CALIFORNIA, Oakland, CA (US)

(72) Inventors: Arash Kheradvar, Irvine, CA (US); Daryl C. Nguyen, Irvine, CA (US)

(21) Appl. No.: 17/088,989

(22) Filed: Nov. 4, 2020

Related U.S. Application Data
(60) Provisional application No. 62/932,735, filed on Nov. 8, 2019.

(51) Int. Cl. A61F 2/24

(52) U.S. Cl. CPC A61F 2/2418; A61F 2220/0075; A61F 2/2433

(57) ABSTRACT

Disclosed herein is a valve system with an original deployed diameter that is suitable for a child and/or young adult and that can accommodate the patient's growth by a one-time balloon expansion to attain an expanded diameter in that patient. This novel valve addresses an unmet need for children with valve dysfunction and to stop valve-related progressive ventricular dysfunction, and ultimately avoid heart failure in children and young adults.

### CROSS-REFERENCES TO RELATED APPLICATIONS

This application is a non-provisional and claims benefit of U.S. Provisional Application No. 62/932,735 filed Nov. 8, 2019, the specification(s) of which is/are incorporated herein in their entirety by reference.

### BACKGROUND OF THE INVENTION

### Field of the Invention

The present invention relates to prosthetic valve systems, namely, a valve system that accommodates a child's growth through a balloon expansion and methods of use. A non-limiting application of such a valve system is to replace a diseased pulmonary valve, which is commonly involved in Congenital Heart Disease (CHD).

### Background Art

Congenital heart defects (CHDs) occur in ˜1% of births in the U.S. and Europe. Presently, it is estimated that at least 1 million children are living with CHD in the U.S. Owing to improved medical and surgical care, it is estimated that 83% of babies born with CHD in the U.S. survive infancy. Right ventricular outflow tract (RVOT) obstruction is frequently present in a variety of patients with CHD such as, but not limited to, pulmonary atresia and tetralogy of Fallot (TOF). The RVOT obstruction in these patients often needs to be surgically relieved within the first few months of life. Although the initial surgical repair technique has progressed over the years, it usually does not provide lifelong correction of the defect. Because the RVOT in infants is small, there is currently no prosthetic valve that can be implanted at the time of initial surgery. Surgery to relieve RVOT obstruction usually involves placing a transannular patch, which results in pulmonary valve regurgitation (PVR) that can lead to progressive RV dilation over time. This can then cause RV dysfunction, which can ultimately lead to RV failure.

It is generally accepted that PVR must be addressed in “suitable candidates.” In asymptomatic patients, indication for transcatheter pulmonary valve (TPV) implantation is based on the presence of: (1) PV regurgitation >20%, indexed end-diastolic RV volume >120-150 ml/m2 BSA, and indexed end-systolic RV volume >80-90 ml/m2 BSA. If any of those are observed in a patient less than about 18 kg, the current strategy is to wait. However, progressive PVR can leave irreversible sequelae on the RV in children as small as 8 to 10 kg.

To avoid the occurrence of RV failure, the Melody™ TPV (Medtronic Inc.) is implanted in these patients using the Medtronic Ensemble delivery system, a 22 French-catheter (Fr) delivery system. Presently, the smallest Melody valve (Melody TPV 20) can only be implanted in a child whose weight is at least 18-20 kg and can only be deployed up to 20 mm in diameter. To avoid RV dysfunction, progressive PVR must be addressed at a younger age, ideally in patients as small as 8-10 kg. While early TPV implantation is potentially critical in preventing RV dysfunction, one should be cognizant of the fact that the child will continue to grow and will eventually require a larger pulmonary valve. Hence, there is an unmet clinical need for TPVs that can accommodate growth of children with progressive PVR whose weight is between 8 and 20 kg.

Other types of heart valve diseases in child and young patients may also require valve replacement to prevent valve dysfunction as the patients grow and their cardiac dimensions enlarge. In some aspects, regurgitation (e.g. backflow), stenosis (e.g. stiffening), or atresia (e.g. malformation) of the tricuspid, pulmonary, mitral, or aortic valve are examples of heart valve diseases in children or young adults that may be treated with valve replacement. The present invention proposes a solution to the unmet clinical need by providing a growth-accommodating prosthetic valve that can replace any of the heart valves and is capable of expanding to meet the size requirements.

### BRIEF SUMMARY OF THE INVENTION

It is an objective of the present invention to provide a novel transcatheter valve whose size can be enlarged for growth-accommodation, as specified in the independent claims. Embodiments of the invention are given in the dependent claims. Embodiments of the present invention can be freely combined with each other if they are not mutually exclusive.

In some aspects, the present invention features a growth-accommodating prosthetic valve comprising a stent and a plurality of leaflets arranged to form a valve. The plurality of leaflets is attached to the stent frame. Each leaflet may include at least one foldable leaflet portion such that the valve has a first radius when said leaflet portion is folded and a second radius when said leaflet portion is unfolded. The second radius is greater than the first radius. Preferably, the leaflets can maintain coaptation when the leaflet portions are folded and unfolded. In some embodiments, the stent frame is expandable to accommodate the second radius of the valve.

In other aspects, the present invention provides a stented valve having an original deployed diameter of 14 mm, suitable for an 8-10 kg child, and accommodates the child's growth by a one-time balloon expansion to attain a diameter of 22 mm in the child whose weight has reached about 20-22 kg. In some embodiments, the stented valve features folded membrane leaflets that can be assembled into an expandable stent to form a trileaflet heart valve whose size can increase with balloon-expansion while keeping its coapting trileaflet shape intact and unharmed. A planar sheet of clinical quality porcine pericardium having a thickness of ˜200 micron is converted from a flat sheet leaflet to a curved 3D surface with the ability to be expanded to a surface of comparable form/shape at a scaled size to form a scalable valve. Leaflet folding of the thin membrane leaflets is based on the conception of folds as creases of 0th order of the geometric continuity since the fold thickness or maximum curvature at the folds is small.

Balloon-expandable stents are rigid but support high radial outward force and have the capacity to be oversized using a balloon to achieve greater precision in expansion size. In some embodiments, the present invention features a balloon-expandable stent comprising cobalt-chromium (CC) alloy, which, compared to stainless steel, is: (1) stronger, making it possible to have thinner struts without decreasing radial strength, which is essential for open-cell stent designs; (2) denser than 316 L stainless steel, which makes the thin-strut stents more radiopaque compared to stainless steel stent; and (3) MRI compatible due to negligible iron content, which makes the CC alloy non-ferromagnetic.

In some embodiments, the present invention features a hybrid stent in which most of the stent has a closed-cell configuration, with certain areas having open-cell conformation. The areas with open-cell configuration allow for maximal balloon expansion to augment diameter from 14 mm to 22 mm. The closed-cell conformation provides strength and rigidity needed to keep the stented valve in the RVOT position. Thus, the novel TPV stent with hybrid design combines the flexibility of an open-cell stent with the stability of a closed-cell stent design.

In other aspects, the growth-accommodating prosthetic valve system may be used in a device for mitigating progressive valve regurgitation in a patient in need thereof. The valve system can be implanted surgically or transcatheterly into said patient to replace the function of a native or prosthetic heart valve, such as the tricuspid, pulmonary, mitral, or aortic valve. The valve system can be deployed using a first inflatable balloon such that the valve is in the first position. When the patient's cardiac dimensions enlarge, the valve system is expanded using a second inflatable balloon such that the valve is in the second position to accommodate the larger cardiac dimensions. It is to be understood that the present invention is not limited to a specific heart valve or disease.

One of the unique and inventive technical features of the present invention is the folded valve design that uniquely allows for augmentation of the TPV's diameter from 14 mm to 22 mm to accommodate the child's growth. In some aspects, the folded valve design features a sector-shaped leaflet comprising at least one foldable leaflet surface such that the leaflet has a first radius when said leaflet surface is folded and a larger, second radius when said leaflet surface is unfolded. Preferably, the leaflet maintains a concave curvature when the leaflet surface is folded and unfolded.

Without wishing to limit the invention to any theory or mechanism, it is believed that the technical feature of the present invention advantageously allows for mitigating the devastating effects of PVR a few years earlier than the current state of the art, thus minimizing the chance of RV dysfunction in children with RVOT abnormalities. This novel technology will address the current lack of options for children with progressive PVR whose weight is between 8 and 20 kg in order to stop progressive RV dilation, and ultimately avoid the occurrence of RV failure in these children. None of the known prior references or work has the unique inventive technical feature of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

This patent application contains at least one drawing executed in color. Copies of this patent or patent application publication with color drawing(s) will be provided by the Office upon request and payment of the necessary fee.

The features and advantages of the present invention will become apparent from a consideration of the following detailed description presented in connection with the accompanying drawings in which:

FIG. 1 shows a non-limiting embodiment of a growth-accommodating stented valve of the present invention. The top row shows a 14 mm transcatheter pulmonary valve (TPV) and the bottom row shows the TPV having a diameter of 22 mm after balloon expansion. As can be seen, the valve keeps its fully coapting trileaflet shape after balloon expansion.

FIG. 2 shows a comparison of the pre-(left) and post-balloon expansion (right) of the TPV. The folded leaflet tissue has been expanded to maintain the coapting trileaflet shape of the valve.

FIG. 3A is a schematic of a leaflet folding process to scale down a full-size leaflet to a smaller one for making a scalable TPV and keep its coapting trileaflet shape intact.

FIG. 3B shows the original-size leaflet on the left for a 22 mm TPV while the scaled down leaflet on the right is for a 14 mm TPV after doing the folding process of FIG. 3A.

FIG. 4 shows an alternative schematic of a leaflet folding process to scale down a full-size leaflet to a smaller one while keeping its coapting trileaflet shape intact.

FIG. 5 shows another alternative schematic of a leaflet folding process to scale down a full-size leaflet to a smaller one while keeping its coapting trileaflet shape intact.

FIG. 6 is yet another alternative schematic of a leaflet folding process to scale down a full-size leaflet to a smaller one while keeping its coapting trileaflet shape intact.

FIGS. 7A-7B show an embodiment of a cobalt-chromium (CC) balloon expandable stent that may be used to form the TPV of the present invention. FIG. 7A shows the stent originally expanded to 14 mm for children as small as 8-10 Kg. FIG. 7B shows the stent, which has been later balloon-expanded to 22 mm diameter to accommodate child growth. The hybrid configuration of a closed- and open-cell pattern in turn allows for strength and stability, and stent conformity, respectively.

### DETAILED DESCRIPTION OF THE INVENTION

Following is a list of elements corresponding to a particular element referred to herein:
100 prosthetic valve
110 stent frame
112 open-cells
114 closed-cells
120 leaflet
121 first side edge
122 second side edge
124 curved edge
126 leaflet dimension
130 foldable leaflet portion
131 bisecting line
133 central folded flap
135 side folded flap
137 segment
141 first position
142 second position
143 first radius
144 second radius
151 first inflatable balloon
152 second inflatable balloon

Relief of right ventricular outflow tract (RVOT) obstruction in infants by either transannular patch or pulmonary valvotomy is usually hampered by long-term morbidity due to RV volume overload secondary to progressive pulmonary valve regurgitation (PVR). Right ventricular and left ventricular dysfunctions, in addition to development of life threatening atrial and ventricular arrhythmias and exercise limitation, are among the most common consequences of RV volume overload. Sadly, in most cases, patients do not show any PVR-related symptoms until the RV dysfunction is fully established. Therefore, timely implantation of a pulmonary valve is crucial to prevent the detrimental effects of PVR. Although progressive PVR must be addressed at a younger age, in patients who are 10-20 kg, the smallest available valve, Melody™ TPV 20, can only be implanted in a child whose weight is at least 20 kg. A major factor that limits Melody™ valve implantation in younger patients is the 22 French-catheter (Fr) size of the delivery system, which is often too large for the smaller venous anatomy in patients less than 20 kg. Another limiting factor is the RVOT diameter in the smaller children, which is too small and commonly unable to accommodate the 20 mm Melody TPV. The present invention provides a novel valve that can be initially deployed at 14 mm and has the ability to accommodate future balloon expansion to achieve 22 mm diameter in a child who has reached a weight of at least 20 kg.

### Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular. Thus, as used in this specification and the appended claims, the singular forms “a,” “an,” and “the” include plural referents unless the context clearly indicates otherwise.

One skilled in the art would recognize that the term “heart” refers to a muscular organ located just behind and slightly left of the breastbone that pumps blood through the network of arteries and veins called the cardiovascular system. The heart has four chambers comprising the right atrium, the right ventricle, the left atrium, and the left ventricle. Additionally, the heart has four valves comprising the mitral valve (located between the atria), tricuspid valve (located between the ventricles), the aortic valve (located between the left ventricle from the aorta), and the pulmonary valve (located between the right ventricle and pulmonary artery).

Each valve in the heart is made up of strong, thin flaps of tissue called leaflets or cusps. As used herein “cusps” or “leaflets” may be used interchangeably. A leaflet may refer to one of the triangular segments of a valve in the heart which opens and closes with the flow of blood. Leaflets open to let blood move forward through the heart during half of the heartbeat. They close to keep blood from flowing backward during the other half of the heartbeat.

As used herein “coapting” refers to one or more leaflets fitting together closely to prevent backwards blood flow.

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject/patient is a human. In one embodiment, the subject is a mammal (e.g., a human) having a disease, disorder or condition described herein. In another embodiment, the subject is a mammal (e.g., a human) at risk of developing a disease, disorder or condition described herein.

Referring now to FIG. 1, the present invention features a growth-accommodating prosthetic valve system (100) comprising a stent (110) and a multitude of leaflets (120) attached to the stent frame (110) to form a valve. In preferred embodiments, the valve can expand from a first position (141) to a second position (142) (FIG. 1 top vs bottom).

Referring to FIGS. 3A and 3B, each leaflet (120) may include at least one foldable leaflet surface (130) such that when said foldable leaflet surface (130) is folded, the valve is in a first position (141), and when said leaflet surface (130) is unfolded, the valve is in a second position (142). Preferably, the leaflets (120) can maintain coaptation when the valve is either in the first position (141) or second position (142). As used herein, coaptation and equivalents thereof refer to the joining or adjusting of parts or surfaces to each other, such as the intersection of the leaflets.

According to some embodiments, each leaflet (120) may have two side edges (121, 122) and a curved edge (124) that form a sector shape. As known in the art, a sector refers to a structure enclosed by two radii of a circle or ellipse and the arc between them. Preferably, the leaflet (120) maintains a concave curvature when the leaflet portion (130) is folded and unfolded.

Various techniques of folding and unfolding the leaflets to obtain the first position (141) and the second radius (144) are demonstrated in FIG. 3A, FIG. 4, FIG. 5, and FIG. 6. In the example shown in the position of FIG. 3A, the at least one foldable leaflet surface (130) of each leaflet (120) may be folded along a line (131) bisecting the leaflet, and between the bisecting line (131) and the curved edge (124) to form a centrally-located folded flap (133). This folded configuration results in a reduction in at least one dimension (126) of the leaflet and forms the valve into the first position (141). Referring to FIGS. 4-6, the at least one foldable leaflet surface (130) of each leaflet (120) may be folded between the curved edge (124) and a line bisecting (131) the leaflet to form a folded flap (135) on each side of the bisecting line. In yet another embodiment, the at least one foldable leaflet surface (130) of each leaflet (120) is folded along a segment (137) of the curved edge These alternate folded configurations results in a reduction in at least one dimension (126) of the leaflet and forms the valve into the first position (141). The dimensions (126) refer to a height and/or width of the leaflet. In some embodiments, the folded flaps are temporarily attached to the leaflet via an adhesive to maintain the folded configuration.

Referring to FIG. 2, when the valve is in the first position (141) (left), the valve can have a first radius (143). When the valve is in the second position (142) (right), the valve can have a second radius (144). For example, the valve has the first radius (143) when the leaflet surface is folded and the second radius (144) when the leaflet surface is unfolded. As shown in FIG. 3B, the second radius (144) is greater than the first radius (143). Preferably, the stent frame (110) can expand to accommodate the second radius (144) of the valve.

In some embodiments, the first radius (143) is about 5 to 8 mm and the second radius (144) is about 9 to 15 mm. In some embodiments, the first radius (143) is about 5 mm. In some embodiments, the first radius (143) is about 6 mm. In some embodiments, the first radius (143) is about 7 mm. In some embodiments, the first radius (143) is about 8 mm. In some embodiments, the first radius (143) is 5 mm, 6 mm, 7 mm, or 8 mm.

In some embodiments, the second radius (144) is about 9 mm. In some embodiments, the second radius (144) is about 10 mm. In some embodiments, the second radius (144) is about 11 mm. In some embodiments, the second radius (144) is about 12 mm. In some embodiments, the second radius (144) is about 13 mm. In some embodiments, the second radius (144) is about 14 mm. In some embodiments, the second radius (144) is about 15 mm. In some embodiments, the second radius (144) is 9 mm, 10 mm, 11 mm, 12 mm, 13 mm, 14 mm, or 15 mm.

In one embodiment, the multitude of leaflets may comprise three leaflets (120) attached to the stent frame (110) to form a pulmonic valve. In another embodiment, the multitude of leaflets may comprise two leaflets (120) attached to the stent frame (110). In yet another embodiment, the multitude of leaflets may comprise four leaflets (120) attached to the stent frame (110).

In some embodiments, the multitude of leaflets (120) may be attached to the stent frame (110). In some embodiments, the multitude of leaflets (120) may be attached to the stent frame (110) through sewing the multitude of leaflets (120) to the stent frame (110). In some embodiments, the multitude of leaflets (120) are sewn along the curved edge (124) such that the curved edge (124) maintains its concavity when attached. In other embodiments, the multitude of leaflets (120) maintain coaptation when sewn onto the stent frame (110).

In some embodiments, the multitude of leaflets (120) may be attached to the stent frame (110) by bonding the multitude of leaflets (120) to the stent frame (110) with using a polymeric material. In some embodiments, the multitude of leaflets (120) are bonded along the curved edge (124) such that the curved edge (124) maintains its concavity when attached. In other embodiments, the multitude of leaflets (120) maintain coaptation when attached to the stent frame (110).

The folding valve design aims to accommodate future valve augmentation without disrupting its multi-leaflet (e.g. trileaflet) form and coaptation profile. To accommodate this, the excessive leaflet tissue must be folded down over the rest of the leaflet and stuck to it using an adhesive that (1) does not harm the tissue; (2) can safely remain glued until the patient reaches the targeted size; and (3) can be smoothly unbonded by balloon expansion without tearing the leaflet tissue. In some embodiments, hydrogel-based wet adhesives such as biocompatible polyethylene glycol (PEG) hydrogel, may be used for this application. In other embodiments, the adhesive may be other bioinspired reversible adhesives for wet surfaces, as well as medical-grade commercially-available adhesives. In other embodiments, bonding through heat or degradable sewing may be used for this application as well.

In one embodiment, the leaflets may be laser-cut from porcine pericardial tissue with a thickness of about 200 microns and then sewn to a stent. Porcine pericardial tissue is a preferred material because it is less prone to infective endocarditis. Furthermore, due to the leaflets' folding design, the porcine pericardium tissue sheet is easier to laser-cut into leaflets. However, it is to be understood that the leaflets are not limited to porcine pericardium tissue.

In other embodiments, the leaflets may be made from a variety of materials with thicknesses varying from about 50-500 microns. In preferred embodiments, the leaflets may be made from a variety of materials with thicknesses varying from about 100 to 300 microns. In some embodiments, the leaflets may be made from a variety of materials with thicknesses varying from about 50-100 microns, or about 100-150 microns, or about 150-200 microns, or about 200-250 microns, or about 250-300 microns, or about 350-400 microns, or about 450-500 microns. The leaflets may comprise a biologic or synthetic material. Non-limiting examples of suitable materials include, but are not limited to, natural membranes, polymer material (natural or synthetic), engineered biological tissue, biological valvular leaflet tissue, pericardial tissue or crosslinked pericardial tissue, other non-pericardial tissue or xenogeneic valve tissue. In one embodiment, the tissue may be procured from human, bovine, equine, ovine, or other animals. In another embodiment, the crosslinked pericardial tissue is crosslinked with a crosslinking agent such as formaldehyde, glutaraldehyde, dialdehyde starch, antibiotics, glyceraldehydes, cyanamide, diimides, diisocyanates, dimethyl adipimidate, neomycin, carbodiimide, epoxy compound, or any mixture thereof.

In some embodiments, the stent frame (110) is expandable. As an example, the stent frame (110) may comprise a unique hybrid combination of an open- and closed-cell configuration (112 and 114 respectively) to accommodate balloon expansion while keeping its strength and stability in the RVOT position. For example, as shown in FIGS. 7A and 7B, the stent frame (110) may comprise alternating portions of open-cells (112) and closed-cells (114). Two major stent design concepts have traditionally been used for cardiovascular purposes: closed- and open-cell stents. The distinction between the two is based on the amount of space between the stent's latticework. Closed-cell stents have a connecting strut for each stent cell with tighter weaves with small free-cell areas, which makes them more rigid and less conformable. Conversely, cells in the open-cell designs are connected through incomplete tines, which increase the free-cell area compared to the closed-cell stents. Open-cell stents are more flexible and provide better conformability compared to closed-cell stents.

In a preferred embodiment, the stent may be laser-cut from a chromium-cobalt alloy. In other embodiments, the stent may be constructed from a variety of other materials suitable for a desired biological application. Non-limiting examples of suitable material include, but are not limited to, shape-memory materials, stainless steel, polymers, plastic, self-expanding Nitinol, thermal shape memory Nitinol, etc.

In some other embodiments, the stent may also be equipped with at least one bioactive agent for biologically inspired applications. Non-limiting examples of the bioactive agent include analgesics/antipyretics, antiasthmatics, antibiotics, antidepressants, antidiabetics, antifungal agents, antihypertensive agents, anti-inflammatories, antineoplastics, antianxiety agents, immunosuppressive agents, antimigraine agents, sedatives/hypnotics, antipsychotic agents, antimanic agents, antiarrhythmics, antiarthritic agents, antigout agents, anticoagulants, thrombolytic agents, antifibrinolytic agents, platelet aggregation inhibitor agents, and antibacterial agents, antiviral agents, antimicrobials, anti-infective agents, or any combination thereof.

In some embodiments, the valve system (100) is collapsible over a delivery catheter to be delivered and implanted inside a body chamber, including but not limited to a heart, or a vessel including but not limited to an artery, a vein or lymphatic system. For example, the valve system (100) may be implantable inside a heart chamber as a replacement of one of the four native heart valves. As demonstrated in FIG. 2, the valve system (100) is twice expandable. A first inflatable balloon (151) is used to deploy the valve into the first position (141) and a second inflatable balloon (152) is used to expand the valve into the second position (142).

According to some embodiments, the present invention provides a method of treating progressive valve regurgitation in a child in need thereof. The method may comprise implanting in said child any one of the prosthetic valves (100) described herein to replace a native valve when said child weighs about 8-10 kg, deploying the valve (100) such that a radius of the valve is the first radius (141), and expanding the valve (100) when said child weighs about 20-22 kg to unfold the leaflet surfaces (130) such that the radius of the valve is the second radius (142). In one embodiment, the valve (100) may be implanted using a transcatheter delivery system. In some embodiments, the valve (100) is deployed and expanded using inflatable balloons (151, 152).

For structural heart interventions, balloons may be made from a variety of materials, including plastics, polymer fibers, and even Kevler® coating (Dupont®, Wilmington, Del.). The balloon material, shape, and dimensions are selected to achieve optimal valve expansion in RVOT of patients as small as 8-10 kg. Referring to FIG. 2, balloon deployment to achieve a diameter of 14 mm and/or balloon expansion to achieve a diameter of 22 mm may be performed with balloon valvuloplasty catheters including, but not limited to, Z-MEDT™ balloons (B. Braun Interventional Systems Inc.) or True Dilation® balloons (Bard Peripheral Vascular, Inc.).

In some embodiments, the present invention may include a delivery system comprising a repositionable 14-Fr transfemoral venous delivery system for balloon-expandable TPV. The transcatheter delivery system can (1) accommodate an inflating balloon; (2) balloon-expand the TPV; and (3) have the ability to turn wide inside the RV to reach the RVOT. This delivery system is preferably smaller than the 22-Fr Medtronic Ensemble™ delivery system currently used to deliver and implant the Melody valve. A 14-Fr delivery system for TPV can significantly expand the number of patients for on-time relief of their progressive PVR. These are the group of smaller patients whose vasculature could not accommodate the bulky 22-Fr Medtronic Ensemble™ delivery system. The delivery system can include a handle to control TPV delivery, implantation, repositioning, and release. Since the distance that the catheter needs to travel from the femoral vein to reach the RVOT in small children is significantly shorter than in adults, the handle is preferably ergonomically optimized for the pediatric interventional cardiologists performing transcatheter pulmonary valve implantation (TPVI) in children.

### Example

The following is a non-limiting example of the present invention. It is to be understood that said example is not intended to limit the present invention in any way. Equivalents or substitutes are within the scope of the present invention.

Implant TPVs in ˜10 kg Minipigs

A growth-accommodating transcatheter pulmonary valve can be implanted in minipigs as small as 8-10 kg and later augmented to be suitable for minipigs weighing 20-22 kg through one-time balloon expansion.

A total of 6 minipigs (3 female and 3 male), weighing approximately 8-10 kg (˜3 months old) is intubated under anesthesia and prepared for TPVI. Femoral vein access is obtained with or without the aid of ultrasound, and a 7-Fr introducer sheath is placed therein. RVOT angiography with biplane fluoroscopy is performed using an angiographic catheter in the RVOT or RV, which provides comprehensive anatomic information. The fluoroscopic projections are adjusted as needed to provide optimal RVOT visualization. Although RVOT obstruction is not anticipated in minipigs, to replicate a real-life TPVI procedure, the RVOT may first be pre-stented with a single stent (covered CP stent, Palmaz or EV3). Pre-stenting can serve as a housing for the TPV and may also prolong the valve life.

The valve is inspected and washed in sterile saline baths before it is hand-crimped over the delivery system's balloon. The delivery system is equipped with a 14 mm balloon, which is used for initial implantation. For valve deployment, the balloon's inflation is rated up to ˜3 to 4 atmospheres. Following deployment, repeat hemodynamics and pulmonary artery angiography is performed to measure RVOT gradient and demonstrate a competent pulmonary valve. Post-dilation of the TPV is typically not necessary although it can be performed safely, as needed. Additional imaging with intracardiac echocardiography (ICE) may also be performed to evaluate TPV function and check for any potential perivalvular leak. Following the TPVI procedure and echocardiographic examination, the animal is carefully observed during the immediate postoperative period for bleeding. Once the animal is stable, it is recovered from anesthesia, and then to the animal housing. Daily care records are maintained for each animal from the day of procedure until the animal grows to 20-22 kg (7-8 months old) and is ready to have its TPV augmented to accommodate its growth.

In about 5 months, when the animals reach the targeted weight of 20-22 Kg, they undergo balloon expansion to augment their implanted 14 mm valve to 22 mm. Balloon valvuloplasty catheters such as, but not limited to, Z-MED™ balloons (B. Braun Interventional Systems Inc., Bethlehem, Pa.) and True Dilation® balloons (Bard Peripheral Vascular, Inc., Temple, Ariz.) may be used. For a second time, the animal is to be anesthetized and intubated according to the IACUC-approved procedures. With the guide of fluoroscopy, the balloon valvuloplasty catheter is delivered through the TPV and inflated up to 4 atmospheres to radially expand the TPV to 22 mm and open up the folded leaflets to form a new, larger trileaflet valve.

Electrocardiograms are monitored continuously. Following the procedure, echocardiography is used to assess valve patency and potential regurgitation, mean pressure gradient, effective orifice area (EOA), and valve motion. After recovery, the animals are moved to the post-operative care unit, and then to the animal housing where daily care records are maintained. After the balloon expansion, each animal is kept for another month to test the performance of the expanded valve using weekly echocardiography. After completing the study, all minipigs are euthanized.

Completion of the preclinical study will be followed by explantation of the heart to inspect the integrity of the prosthesis, potential presence of vegetation as indication of infective endocarditis, and to study the valve histology for presence of inflammation and calcification.

Post-Explant Analysis of the TPVs' Microstructure

Conventional histological assays, immunohistochemistry (IHC), and advanced microscopy are used to quantify potential calcification, leaflet thickening, inflammation, and fibrosis. As needed, special stains, including periodic acid-Schiff, Giemsa, Gram, Grocott-Gomori methenamine-silver nitrate, and Warthin-Starry silver stains can be used to detect bacteria and fungi. Changes in the valve matrix are also analyzed by IHC. ECM protein content (collagen, elastin, and GAG) are biochemically quantified and compared to the control intact porcine pericardial tissues.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A growth-accommodating prosthetic valve system (100) comprising:
a) an expandable stent frame (110); and
b) a multitude of leaflets (120) attached to the stent frame (110) to form a valve, wherein the valve is expandable from a first position (141) to a second position (142).

2. A growth-accommodating prosthetic valve system (100) comprising:
a) an expandable stent frame (110); and
b) a multitude of leaflets (120) attached to the stent frame (110) to form a heart valve, wherein each leaflet (120) has at least one foldable leaflet surface (130) such that when said foldable leaflet surface (130) is folded, the valve is in a first position (141), and when said leaflet surface (130) is unfolded, the valve is in a second position (142).

3. The valve system (100) of claim 2, wherein the folded leaflet surface (130) forms a flap that is temporarily attached to the leaflet via an adhesive to maintain the folded configuration.

4. The valve system (100) of claim 2, wherein the multitude of leaflets (120) maintain coaptation when the valve is either in the first position (141) or second position (142).

5. The valve system (100) of claim 4, wherein each leaflet (120) has two side edges (121, 122) and a curved edge (124) that form a sector shape.

6. The valve system (100) of claim 5, wherein the at least one foldable leaflet surface (130) of each leaflet (120) is folded along a line (131) bisecting the leaflet, and between the bisecting line (131) and the curved edge (124) to form a centrally-located folded flap (133), thereby reducing at least one dimension (126) of the leaflet and forming the valve into the first position (141).

7. The valve system (100) of claim 5, wherein the at least one foldable leaflet surface (130) of each leaflet (120) is folded between a line bisecting (131) the leaflet and the curved edge (124) to form a folded flap (135) on each side of the bisecting line, thereby reducing at least one dimension (126) of the leaflet and forming the valve into the first position (141).

8. The valve system (100) claim 2, wherein each leaflet (120) is folded along a segment (137) of the curved edge, thereby reducing at least one dimension (126) of the leaflet and forming the valve into the first position (141).

9. The valve system (100) of claim 8, wherein when the valve is in the first position (141), the valve has a first radius (143), wherein when the valve is in the second position (142), the valve has a second radius (144), wherein the first radius (143) is smaller than the second radius (144).

10. The valve system (100) of claim 9, wherein the first radius (143) is about 7±2 mm and the second radius (144) is about 11±3 mm.

11. The valve system (100) of claim 2, wherein the leaflets (120) comprise a biologic or synthetic material.

12. The valve system (100) of claim 2, wherein the leaflets (120) have a thickness of at least about 50 microns.

13. The valve system (100) of claim 2, wherein the stent frame (110) is expandable to accommodate the second position (142) of the valve.

14. The valve system (100) of claim 2, wherein the stent frame (110) comprises open-cell portions (112) and closed-cell portions (114).

15. The valve system (100) of claim 2, wherein the valve system (100) is collapsible over a delivery catheter to be delivered and implanted inside a body chamber.

16. The valve system (100) of claim 2, wherein the valve system (100) is implantable inside a heart chamber as a replacement of one of the four native heart valves.

17. The valve system (100) of claim 2, wherein the valve system (100) is twice expandable to deploy the valve into the first position (141) using a first inflatable balloon (151) and to expand the valve into the second position (142) using a second inflatable balloon (152).

18. A device for mitigating progressive valve regurgitation in a patient in need thereof, said device comprising a growth-accommodating prosthetic valve system (100) according to claim 2, wherein said valve system (100) is implanted surgically or transcatheterly into said patient to replace the function of a native or prosthetic heart valve, wherein the valve system (100) is deployed using a first inflatable balloon (151) such that the valve is in the first position (141), wherein when said patient's cardiac dimensions enlarge, the valve system (100) is expanded using a second inflatable balloon (152) such that the valve is in the second position (142) to accommodate the larger cardiac dimensions.

19. A sector-shaped leaflet (120) comprising at least one foldable leaflet surface (130) such that the leaflet has a first radius (143) when said leaflet surface (130) is folded and a second radius (144) when said leaflet surface (130) is unfolded, wherein the second radius (144) is larger than the first radius (143).

20. The leaflet (120) of claim 19, wherein the leaflet (120) maintains a concave curvature when the leaflet surface (130) is folded and unfolded.
